(19) United States
(12) Patent Application Publication
(10) Pub. No.: US 2025/0152153 A1
(43) Pub. Date: May 15, 2025

(54) SURGICAL RETRACTOR

(71) Applicant: Shukla Medical, St. Petersburg, FL (US)

(72) Inventors: Adam GOSIK-WOLFE, Auburndale, FL (US); Austin STROH, Lutz, FL (US); Aabhas JAIN, Baltimore, MD (US)

(73) Assignee: Shukla Medical, St. Petersburg, FL (US)

(21) Appl. No.: 18/948,215

(22) Filed: Nov. 14, 2024

Related U.S. Application Data
(60) Provisional application No. 63/598,576, filed on Nov. 14, 2023.

(51) Int. Cl. A61B 17/02

(52) U.S. Cl. CPC A61B 17/0206

(57) ABSTRACT

A surgical retractor including a first retractor arm, a second retractor arm, and a third retractor arm coupled with the first retractor arm and the second retractor arm to move in tandem therewith is disclosed. The first retractor arm and the second retractor arm pivot about a single axis of the surgical retractor.

### FIELD OF THE INVENTION

The present invention relates generally to devices and methods for surgical retractors. In particular, the subject disclosure relates to a multi-prong surgical retractor.

### BACKGROUND OF THE DISCLOSURE

Surgeons commonly use surgical retractors to hold surgical incisions open, providing access to internal bodily tissues. Traditional surgical retractors generally fall into two categories: single-finger and two-finger retractors. Such single-finger retractors must be manually held by the surgeon (or user) throughout the surgical procedure, which can divert the surgeon's attention from other critical tasks. Two-finger retractors, on the other hand, feature a pair of retractor arms and are typically self-locking. While these two-finger retractors may allow the retractor arms to be locked in place without requiring the surgeon's continuous hold, they only retract the incision site in one direction.

Therefore, there is still a need and desire to have devices and methods that address the limitations of traditional surgical retractors.

### BRIEF SUMMARY OF THE DISCLOSURE

In accordance with an exemplary embodiment of the subject disclosure

there is provided a surgical retractor comprising: a first retractor arm, a second retractor arm; a third retractor arm coupled with the first retractor arm and the second retractor arm to move in tandem therewith.

According to an aspect, the first retractor arm and the second retractor arm pivot about a single axis. According to another aspect, the surgical retractor comprises a retractor body operatively connected to the third retractor arm. According to another aspect, the retractor body includes a pivot pivotably connected to the first retractor arm and the second retractor arm. According to another aspect, the retractor body is configured to receive the third retractor arm therein. According to another aspect, the retractor body includes a ratcheting mechanism operatively engaging the third retractor arm. According to another aspect, the first, second, and third retractor arms are movable relative to the retractor body.

According to an aspect, a first pivot arm connected to the first retractor arm and the third retractor arm, and a second pivot arm connected to the second retractor arm and the third retractor arm. According to another aspect, the first, second, and third retractor arms are movable between the first position and second position spaced from the first position. According to another aspect, the first, second, and third retractor arms are movable between the first position and second position spaced from the first position. According to another aspect, each of the first, second, and third retractor arms comprises a modular finger extending therefrom.

In accordance with another exemplary embodiment of the subject disclosure there is provided a surgical retractor comprising a retractor body that includes a ratcheting mechanism; a first retractor arm pivotably connected to the retractor body and including a first finger extending therefrom; a second retractor arm pivotably connected to the retractor body and including a second finger extending therefrom; a third retractor arm operatively coupled with the first and second retractor arms to move in tandem therewith and including a third finger extending therefrom, wherein the first retractor arm and the second retractor arm pivot about a single axis; a first pivot arm connected to the first retractor arm and the third retractor arm; and a second pivot arm connected to the second retractor arm and the third retractor arm; wherein the retractor body is configured to operatively engage the third retractor arm.

Other features and advantages of the subject disclosure will be apparent from the following more detailed description of the exemplary embodiments.

### BRIEF DESCRIPTION OF THE SEVERAL VIEWS OF THE DRAWINGS

The foregoing summary, as well as the following detailed description of the exemplary embodiments of the subject disclosure, will be better understood when read in conjunction with the appended drawings. For the purpose of illustrating the present disclosure, there are shown in the drawings exemplary embodiments. It should be understood, however, that the subject application is not limited to the precise arrangements and instrumentalities shown.

FIGS. 1 and 2 are perspective views of a surgical retractor in accordance with an exemplary embodiment of the subject disclosure;

FIG. 3 is a top plan view of the surgical retractor of FIG. 1;

FIG. 4 is a perspective view of a first retractor arm of the surgical retractor of FIG. 1;

FIG. 5 is a perspective view of a second retractor arm of the surgical retractor of FIG. 1;

FIG. 6 is a top plan view of a third retractor arm and a retractor body of the surgical retractor of FIG. 1;

FIGS. 7-8 show perspective views of the third retractor arm of the surgical retractor of FIG. 1;

FIG. 9 shows a perspective view of a first pivot arm and a second pivot arm of the surgical retractor of FIG. 1;

FIG. 10 is a rear perspective view of the first pivot arm and the second pivot arm of the surgical retractor of FIG. 1;

FIG. 11 is an exploded view of a fastener, a mounting bracket and a retaining ring of the surgical retractor of FIG. 1;

FIGS. 12-13 show perspective views of the mounting bracket of the surgical retractor of FIG. 1;

FIG. 14 is a perspective view of the retractor body of the surgical retractor of FIG. 1;

FIGS. 15-17A and 17B show perspective views of a ratcheting mechanism and components of the surgical retractor of FIG. 1;

FIG. 18 is an enlargement view of a second finger mount of the surgical retractor of FIG. 1;

FIG. 19 is a perspective view of an exemplary modular finger of the surgical retractor of FIG. 1;

FIGS. 20A-C are a series of views of a surgical retractor in accordance with an exemplary embodiment of the subject disclosure, illustrating the operation thereof;

FIGS. 21-22 are perspective views of a surgical retractor in accordance with an exemplary embodiment of the subject disclosure;

FIG. 23 is a top plan view of the surgical retractor of FIG. 21;

FIG. 24 is a perspective view of a first retractor arm of the surgical retractor of the surgical retractor of FIG. 21;

FIG. 25 is a perspective view of a second retractor arm of the surgical retractor of FIG. 21;

FIG. 26 is a perspective view of a third retractor arm and a retractor body of the surgical retractor of FIG. 21;

FIGS. 27-28 show perspective views of the third retractor arm of the surgical retractor of FIG. 21;

FIGS. 29-30 show perspective views of a first pivot arm and a second pivot arm of the surgical retractor of FIG. 21;

FIGS. 31-35 are perspective views and an exploded view of a finger mount of the surgical retractor of FIG. 21;

FIG. 36 is an exploded view of the finger mount of the surgical retractor of FIG. 21;

FIG. 37 is a perspective view of the finger mount of the surgical retractor of FIG. 21;

FIGS. 38-42 show various views of a ratchet housing of the surgical retractor of FIG. 21; and

FIG. 43 is a perspective view of an exemplary modular finger of the surgical retractor of FIG. 21.

### DETAILED DESCRIPTION OF THE DISCLOSURE

Reference will now be made in detail to the various exemplary embodiments of the subject disclosure illustrated in the accompanying drawings. Wherever possible, the same or like reference numbers will be used throughout the drawings to refer to the same or like features. It should be noted that the drawings are in simplified form and are not drawn to precise scale. Certain terminology is used in the following description for convenience only and is not limiting. Directional terms such as top, bottom, left, right, above, below and diagonal, are used with respect to the accompanying drawings. The term “distal” shall mean away from the center of a body. The term “proximal” shall mean closer towards the center of a body and/or away from the “distal” end. The words “inwardly” and “outwardly” refer to directions toward and away from, respectively, the geometric center of the identified element and designated parts thereof. Such directional terms used in conjunction with the following description of the drawings should not be construed to limit the scope of the subject application in any manner not explicitly set forth. Additionally, the term “a,” as used in the specification, means “at least one.” The terminology includes the words above specifically mentioned, derivatives thereof, and words of similar import.

“About” as used herein when referring to a measurable value such as an amount, a temporal duration, and the like, is meant to encompass variations of ±20%, ±10%, ±5%, ±1%, or ±0.1% from the specified value, as such variations are appropriate.

“Substantially” as used herein shall mean considerable in extent, largely but not wholly that which is specified, or an appropriate variation therefrom as is acceptable within the field of art. “Exemplary” as used herein shall mean serving as an example.

Throughout the subject application, various aspects thereof can be presented in a range format. It should be understood that the description in range format is merely for convenience and brevity and should not be construed as an inflexible limitation on the scope of the subject disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Furthermore, the described features, advantages and characteristics of the exemplary embodiments of the subject disclosure may be combined in any suitable manner in one or more embodiments. One skilled in the relevant art will recognize, in light of the description herein, that the subject disclosure can be practiced without one or more of the specific features or advantages of a particular exemplary embodiment. In other instances, additional features and advantages may be recognized in certain embodiments that may not be present in all exemplary embodiments of the present disclosure.

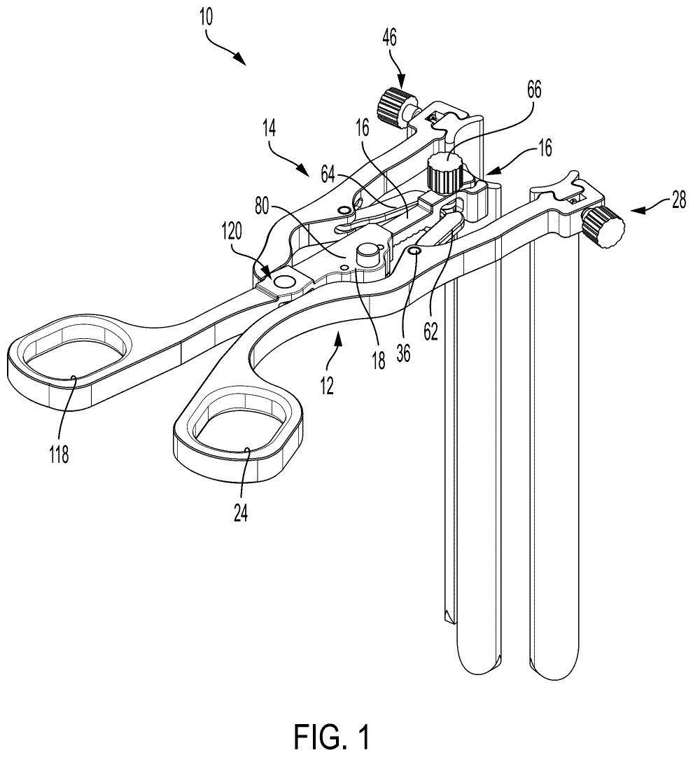

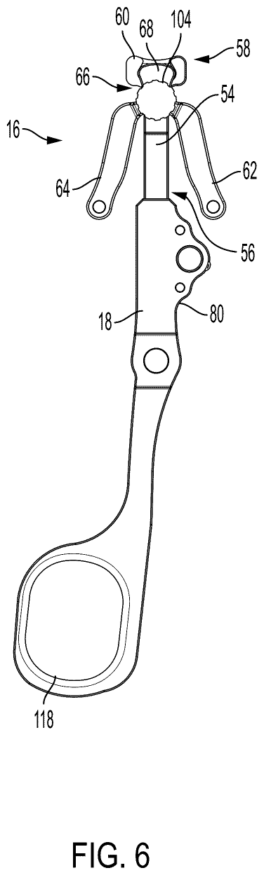

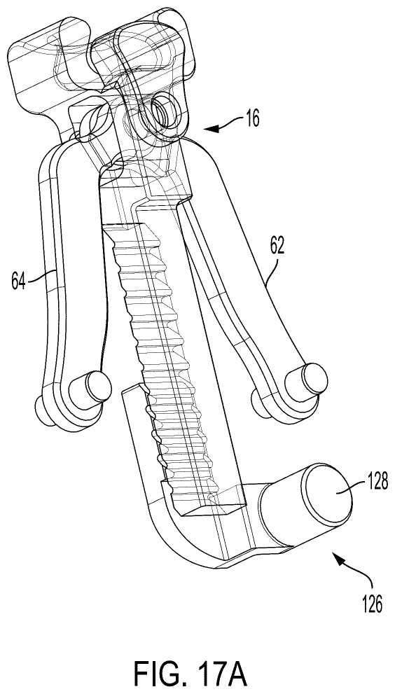

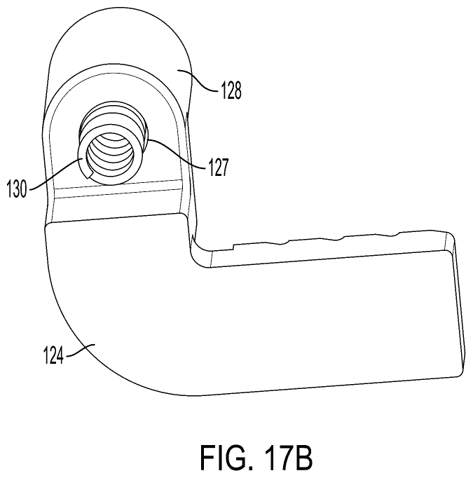

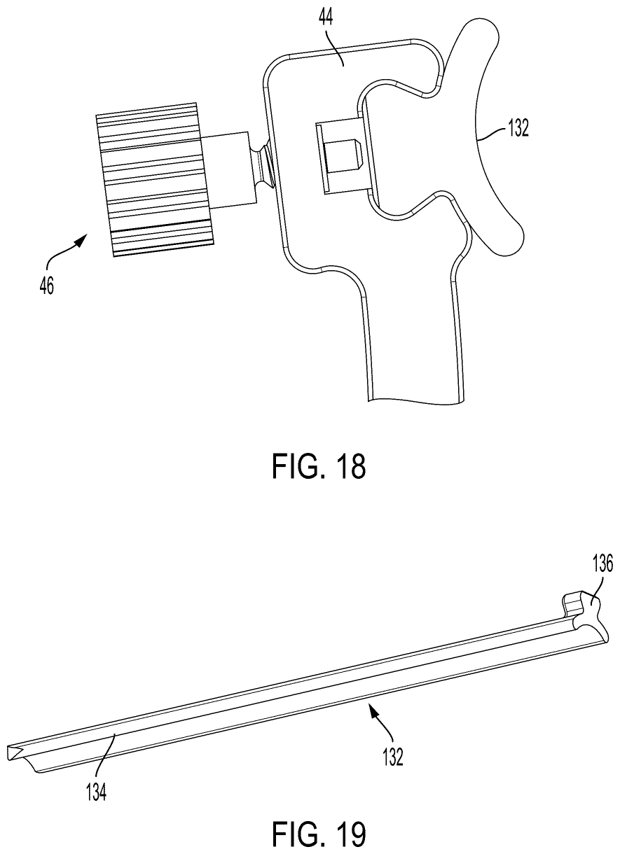

Referring to the drawings, FIGS. 1-20 illustrate a surgical retractor 10 in accordance with a first exemplary embodiment of the subject disclosure. The surgical retractor 10 includes a first retractor arm 12, a second retractor arm 14, a third retractor arm 16, and a retractor body 18.

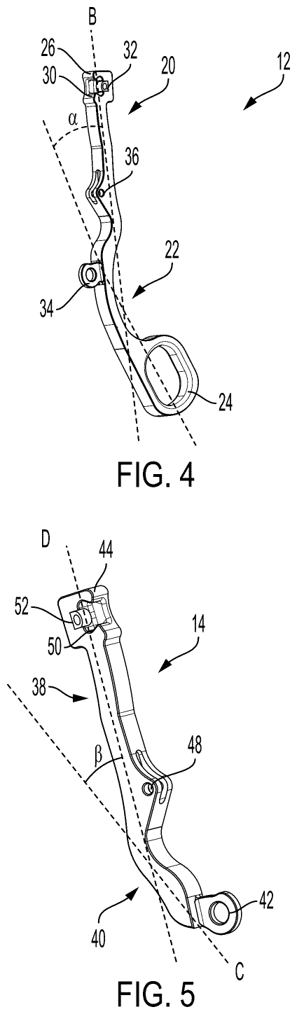

The first retractor arm 12 is configured as best shown in FIGS. 1-4. The first retractor arm 12 is an elongated shaft having a substantially linear portion 20 and a non-linear portion 22 extending from the linear portion 20. The substantially linear portion 20 forms the distal end of the first retractor arm 12 and the non-linear portion 22 forms the proximal end of the first retractor arm 12. The non-linear portion 22 has a longitudinal axis A at an angle α of about 30 to 45 degrees including 25, 35, 40 and 50 degrees, relative to a longitudinal axis B of the linear portion 20 (as shown in FIG. 4). The non-linear portion 22 has a curved portion which defines the angle α of its longitudinal axis relative to the linear portion 20. Terminating about its proximal end, the non-linear portion 20 includes a first finger loop 24.

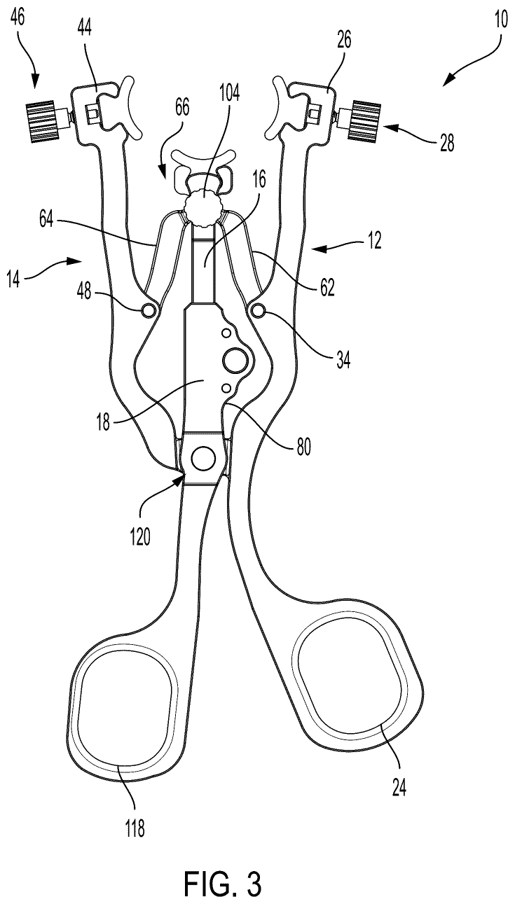

Referring to FIGS. 3-4, the substantially linear portion 20 includes a first finger mount 26 and a first set screw 28 about its distal end. The first finger mount 26 is configured as a dovetailed recess 30 having an open medial side or a medial open face, and a threaded through hole 32 about its lateral side or lateral side wall for operatively engaging or receiving the first set screw 28 therein.

The substantially linear portion 20 also includes a first pivot 34 of the first retractor arm about its medial side, and a second pivot 36 of the first retractor arm about its medial side between the first pivot 34 and the first finger mount 26. The first pivot 34 is positioned between the second pivot 36 and the first finger loop 24. The first pivot 36 is also positioned about a midpoint of the first retractor arm 12 or alternatively at an intersection of the substantially linear portion 20 and the non-linear portion 22.

The second retractor arm 14 is configured as best shown in FIGS. 1, 3, and 5. The second retractor arm 14 is an elongated shaft having a substantially linear portion 38 about its distal end and a bent portion 40 extending from the linear portion 38 (as shown in FIG. 3). The substantially linear portion 38 forms the distal end of the second retractor arm 14 and the bent portion 40 forms the proximal end of the second retractor arm 14. The bent portion 40 has a longitudinal axis C at an angle β of about 25 to 45 degrees including 30, 35 and 40 degrees, relative to a longitudinal axis D of the linear portion 38 (as shown in FIG. 5). Terminating about its proximal end, the bent portion 40 includes a first pivot 42 of the second retractor arm. The first pivot 42 extends medially or alternatively is positioned about a medial side of the bent portion of the second retractor arm.

The substantially linear portion 38 includes a second finger mount 44, a second set screw 46, and a second pivot 48 (as shown in FIGS. 3, 5, and 18). The second pivot 48 is positioned between the second finger mount 44 and the first pivot 42, and substantially about a midportion of the second retractor arm. The second pivot 48 is also positioned about a medial side of the second retractor arm.

The second finger mount 44 is configured as best shown in FIG. 5 and positioned about the distal end of the second retractor arm. The second finger mount 44 is configured as a dovetailed recess 50 having an open medial side or a medial open face and a threaded through hole 52 about its lateral side or lateral side wall for operatively engaging or receiving the second set screw 46 therein.

The third retractor arm 16 is configured as best shown in FIGS. 1, 2, 6-8, and 15-17A and 17B. The third retractor arm 16 includes an elongated body 54 having a proximal end 56 and a distal end 58. The third retractor arm 16 also includes a third finger mount 60, a first pivot arm 62, a second pivot arm 64, a fastener 66, and a mounting bracket 68.

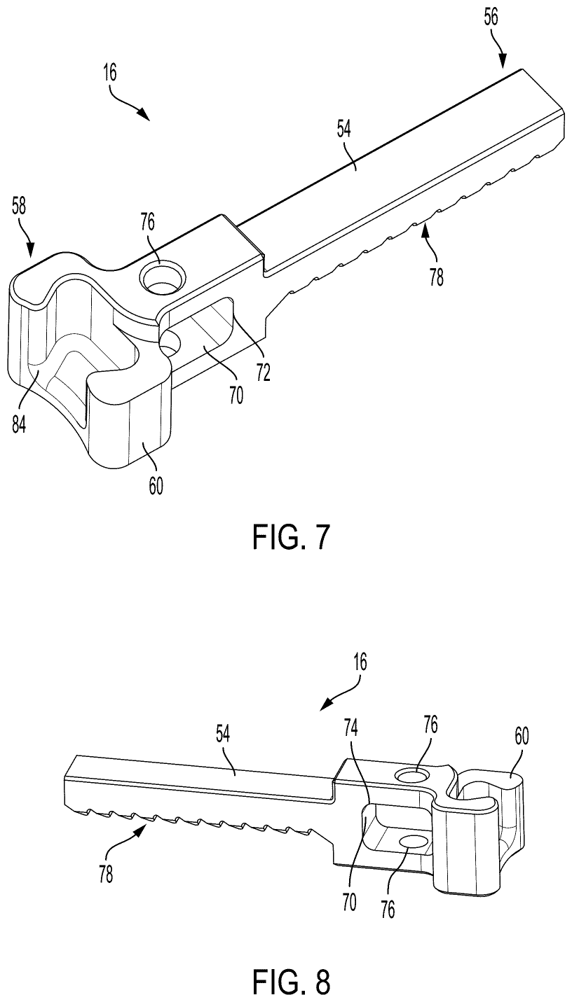

Referring to FIGS. 7 and 8, the elongated body 54 includes a through cavity 70 for receiving, in accordance with an exemplary aspect, the first and second pivot arms therein. The through cavity 70 includes open lateral sides 72, 74 and a through hole 76 extending through the elongated body 54 and through cavity 70 transverse to the open lateral sides or alternatively transverse to a longitudinal axis of the elongated body.

The elongated body 54 further includes a linear ratchet mechanism 78 for operatively engaging a ratchet housing 80 (FIG. 14) of the retractor body 18, and extends from the distal end of the elongated shaft forming the cavity 70. The linear ratchet mechanism 78 forms the bottom or inferior surface of the proximal end 56 of the elongated body 54, or alternately one of the top, bottom, or side surfaces of the proximal end of the elongated body for operatively engaging a correspondingly configured ratcheting mechanism of the ratchet housing (as further discussed below). The linear ratchet mechanism 78 includes teeth configured to operatively engage corresponding ratchet teeth of the ratchet housing 80.

The third finger mount 60 is configured as shown in FIGS. 7-8 and is positioned about the distal end of the third retractor arm e.g., about a most distal end of the elongated body 54. The third finger mount 60 is further configured as a dovetailed recess 84 having an open distal side or a distal open face.

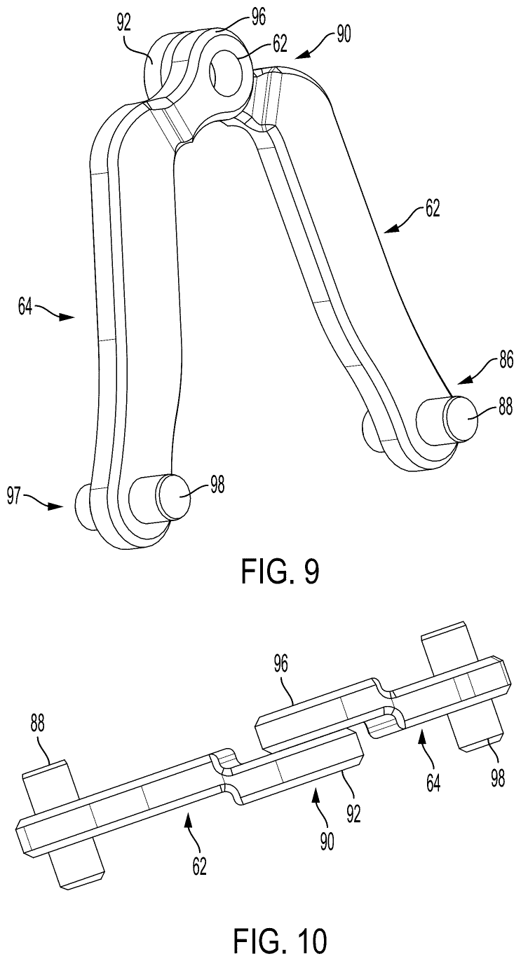

The first and second pivot arms 62 and 64 are configured as best shown in FIGS. 9-10. The first pivot arm 62 includes a proximal end 86 having a pivot connection 88 and a distal end 90 having another pivot connection 92. The pivot connection 92 can be configured as a through hole for receiving a pivot shaft or a threaded shaft of the fastener 66 (as further discussed below) therethrough. The pivot connection 92 can also be configured to be offset from a major plane of a main body portion of the first pivot arm 62. The pivot connection 88 can be configured as a shaft or a pair of opposing shafts for operatively engaging a corresponding pivot connection.

The second pivot arm 64 is configured substantially the same as the first pivot arm 62, for example a substantially mirrored configuration except for its pivot connection 96 about its distal end being offset from a major plane of a main body portion of the second pivot arm 64 in an opposite direction as the offset of the pivot connection 92 of the first pivot arm. The second pivot arm also includes pivot connection 98 about its proximal end, similarly configured as pivot 88.

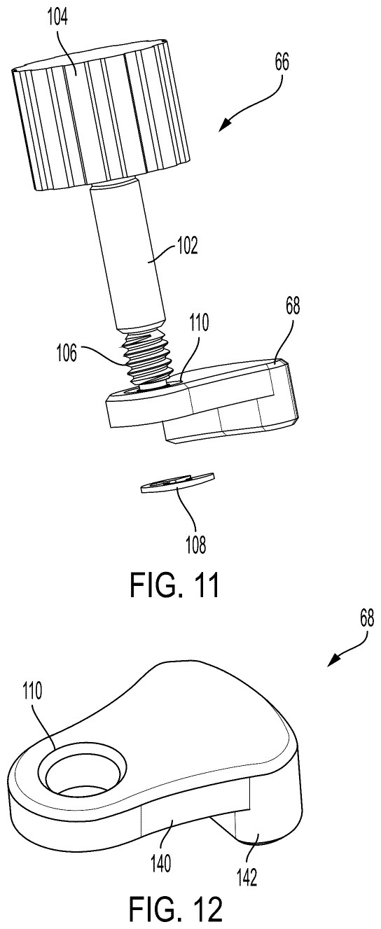

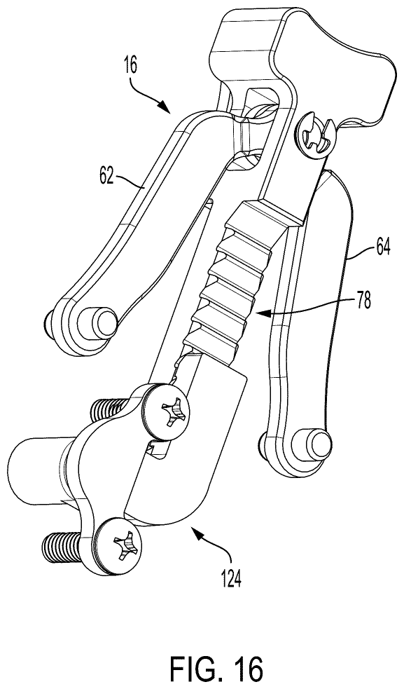

FIG. 11 illustrates the fastener 66 in accordance with the exemplary embodiment. The fastener can be a clamping fastener or clamp for operative engaging the third finger within the third finger mount 60, and the first and second pivot arms. The fastener 66 includes a threaded shaft or pivot shaft 102 having a knob 104, a threaded rod 106, and a retaining clip 108. The threaded shaft 102 is sized and configured to pass through the through hole 110 of the mounting bracket 68 and the through hole 76 of the elongated body 54 forming part of the pivot connection with the first and second pivot arms. That is, the threaded shaft 102 e.g., a female threaded shaft, passes through the through holes of the first and second pivot arms retaining the first and second pivot arms within the cavity 70. The threaded shaft 102 is retained on the elongated body 54 via the threaded rod 106 e.g., a male threaded rod, and the retaining clip 108 about an opposite side of the cavity from which the knob 104 is positioned, as best shown in FIG. 16.

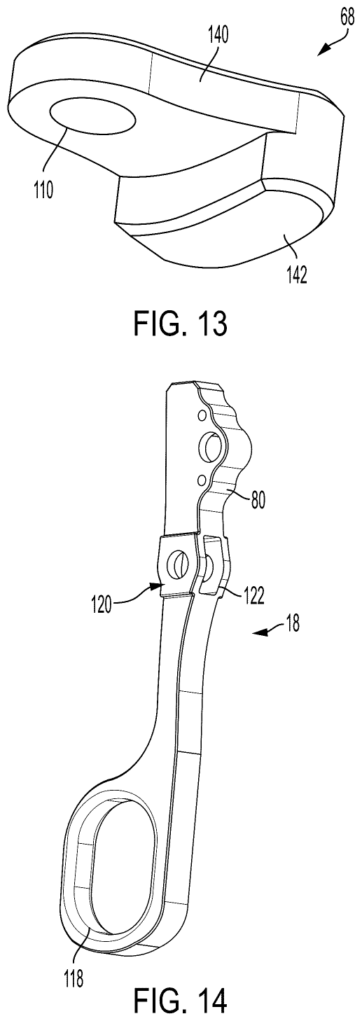

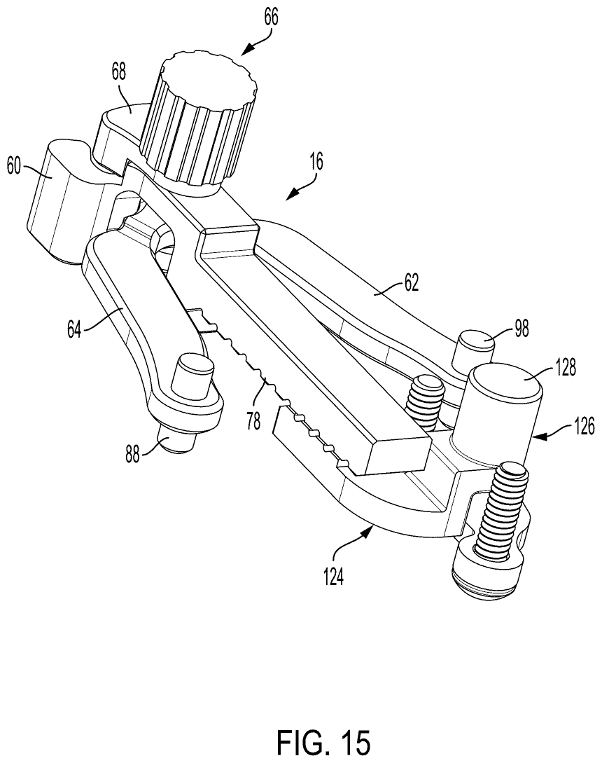

The mounting bracket 68 is configured as best shown in FIGS. 12 and 13. The mounting bracket 68 has a main body 140 that includes the through hole 110 and an elbow 142 correspondingly configured to seat within the third finger mount 60. The through hole 110 is sized and positioned to align with the through holes 76 of the elongated body and receive the threaded shaft 102 therein when assembled with the elongated body 54, as best shown in FIGS. 15 and 16.

The retractor body 18 is configured as best shown in FIGS. 1-3, and 14. The retractor body 18 includes the ratchet housing 80 about its distal end and a second finger loop 118 about its proximal end. The ratchet housing 80 is positioned about a distal end of the retractor body terminating at a point about a pivot connection 120 (as shown in FIGS. 1-3 and 14).

The pivot connection 120 has open lateral and medial sides and is

positioned about a midpoint of the retractor body 18 or alternatively at an intersection of the ratchet housing 80 and the proximal end of the retractor body. The pivot connection 120 includes a through hole transverse to the open lateral and medial sides of the pivot connection, or transverse to a longitudinal axis of the retractor body, for receiving a pivot rod or pivot connection therethrough and operatively engaging the first pivots 34, 42 of the first and second retractor arms. The first pivots 34, 42 of the first and second retractor arms reside within a cavity 122 formed by the pivot connection 120.

As shown in FIGS. 15-17A and 17B, the ratchet housing 80 also includes a corresponding ratcheting mechanism 124 for operatively engaging the linear ratchet mechanism 78. The ratcheting mechanism 124 includes teeth for operatively engaging corresponding ratchet teeth of the linear ratchet mechanism 78. The ratcheting mechanism 124 also includes a release lever 126 having a button 128 to allow a user to lever the ratcheting mechanism 124 away from and out of engagement with the ratchet mechanism so as to allow the user to move the surgical retractor from the retracted position to a non-retracted position e.g., moving the ratchet mechanism relative to the ratcheting mechanism.

A biasing member 130 (e.g., a compression spring, a coil, an elastomer, and the like) is housed within a cavity 127 of the release lever 126 to bias the ratcheting mechanism 124 towards engagement with the linear ratchet mechanism 78.

The foregoing exemplary embodiment illustrates the second finger loop 118 extending from the ratchet housing 80.

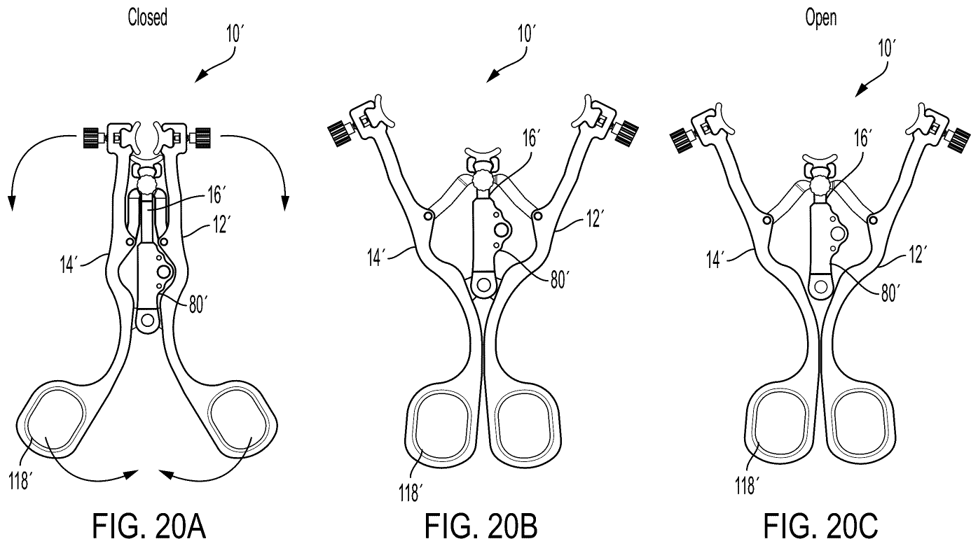

Referring to FIG. 20, in accordance with a second exemplary embodiment of the subject disclosure, the second retractor arm 14′ of the surgical retractor 10′ can be configured as a mirror image or a substantially mirrored image of the first retractor arm 12′. In this embodiment, third retractor arm 16′ and the ratchet housing 80′ are as described above for third retractor arm 16 and ratchet housing 80, except that the second finger loop 118′ no longer extends from the ratchet housing. All other aspects of this second exemplary embodiment are similarly configured as the first exemplary embodiment of the surgical retractor 10.

FIG. 19 illustrates an exemplary modular finger 132 applicable to the first, second, and third retractor arms. That is, finger 132 is configured to seat within the recesses 30, 50, 84 of the first, second, and third retractor arms. The finger 132 is an elongated finger having a tail joint 136 correspondingly configured to seat within the dovetail recess of the first, second or third retractor arms. The elongated finger is also configured to have a transverse cross-section that is substantially C-shaped or curved. That is, the main body 134 of the finger is curved.

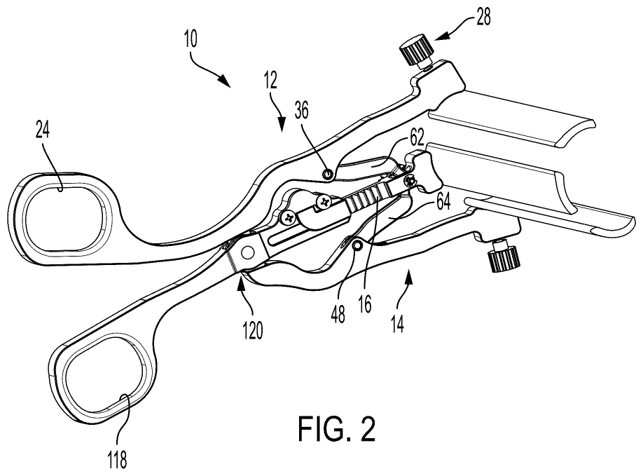

Referring back to FIGS. 1 and 2, the first retractor arm 12 is connected to the retractor body 18 about its first pivot 34 and the second retractor arm 14 is connected to the retractor body 18 about its first pivot 42. That is the first and second retractor arms are pivotably connected to the pivot connection 120 for pivoting about a single axis defined thereby. The third retractor arm 16 is operatively connected to the retractor body 18 and more particularly operatively engages the ratchet housing 80. The first pivot arm 62 has its distal end pivotably connected to the third retractor arm 16 about its pivot connection 92 and its proximal end 86 pivotably connected to the second pivot 36 of the first retractor arm 12. The second pivot arm 64 has its distal end pivotably connected to the third retractor arm 16 about its pivot connection 96 and its proximal end 97 pivotably connected to the second pivot 48 of the second retractor arm 14. Configured as such, each of the first, second, and third retractor arms are interconnected or coupled such that movement of the first retractor arm and/or the second retractor arm drives movement of the third retractor arm. Each of the first, second and third retractor arms can have a finger seated within its respective finger mount.

In operation (of the second exemplary embodiment), owing to the configuration and structure of the first, second and third retractor arms, the surgical retractor 10′ moves between a first position or closed position and a second position or retracted position or open position. Due to the interconnected or coupled configuration of the first, second and third retractor arms, each arm moves simultaneously or in unison as a single unit. Specifically, when the user squeezes the first and second finger loops together (as shown in the embodiment of FIG. 20A-C), the first and second retractor arms 12′ and 14′ pivot about a single axis of the pivot. During movement from the closed position to the open position, the first and second retractor arms rotate outwardly or away from each other, which thereby causes or drives movement of the first and second pivot arms which causes or drives movement of the third retractor arm within the retractor housing thus retracting tissue in a plurality of directions during use, e.g., in the horizontal direction (or medial lateral direction) and the vertical direction (or proximal distal direction) when viewed as shown in FIGS. 20A-C. In the open position or retracted position, the surgical retractor is held in position by the ratcheting mechanism.

Referring back to the operation of the surgical retractor in connection with the first exemplary embodiment, the movement of the first, second, and third retractor arms 12, 14, and 16 is regulated by the biasing member 130 in the ratcheting mechanism 124. When the user squeezes or applies pressure to the first and second finger loops 24 and 118, the teeth of the linear ratchet mechanism 78 interact or engage with the teeth of the ratcheting mechanism 124. The biasing member 130 provides an opposing tension (or force) to maintain this interaction or engagement, allowing the linear ratchet mechanism 78 to move when sufficient force or pressure is applied to first and second finger loops 24 and 118, overcoming the opposing tension, while preventing reverse movement owing to the ratchet mechanism.

To disengage the surgical retractor 10 from the open position, the user presses the button 128 applying a force to the release lever 126. Depressing the release lever levers the ratcheting mechanism 124 away from the linear ratchet mechanism 78 disengaging the ratchet teeth of the respective ratcheting mechanism and linear ratchet mechanism. Upon disengagement of the ratchet mechanism 124, the user can move the retractor arms (and the surgical retractor 10) back to the closed position.

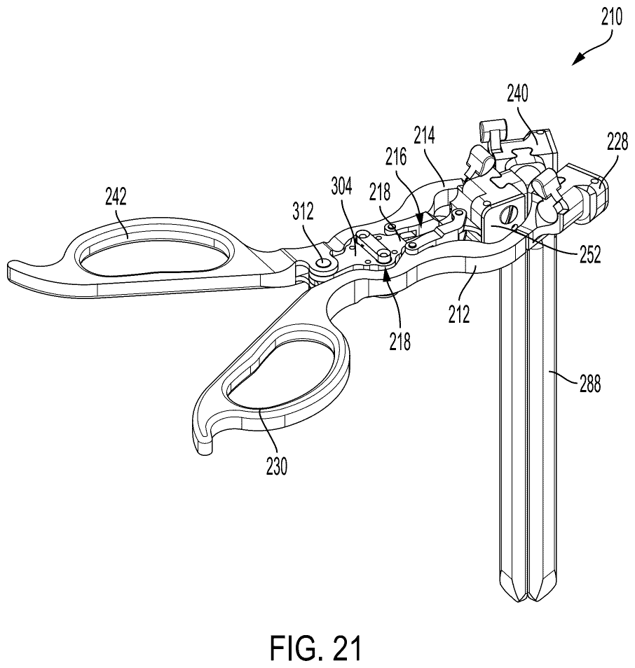

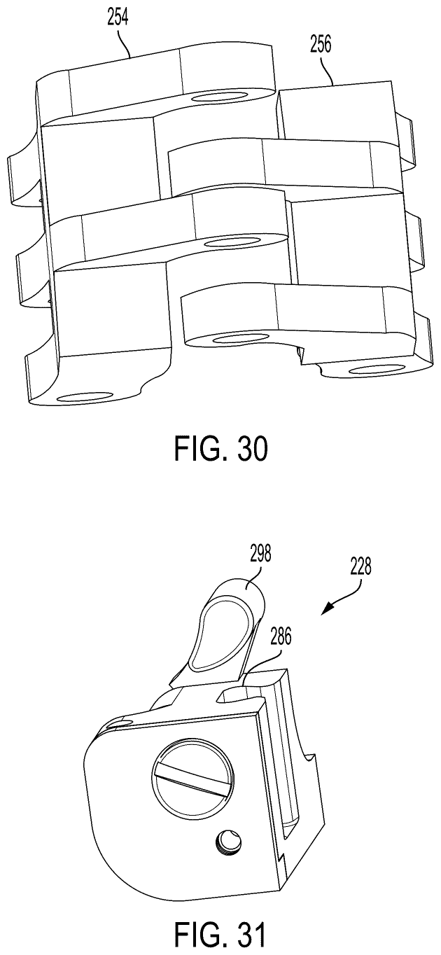

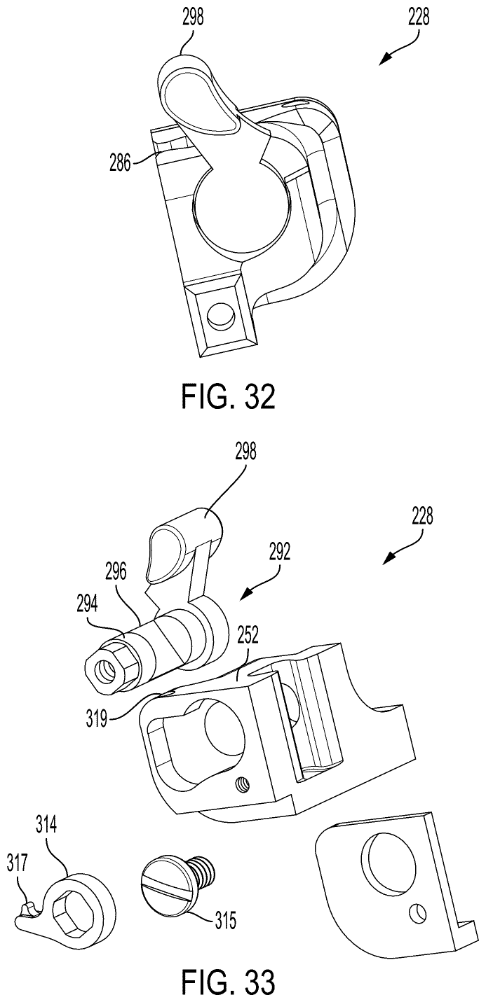

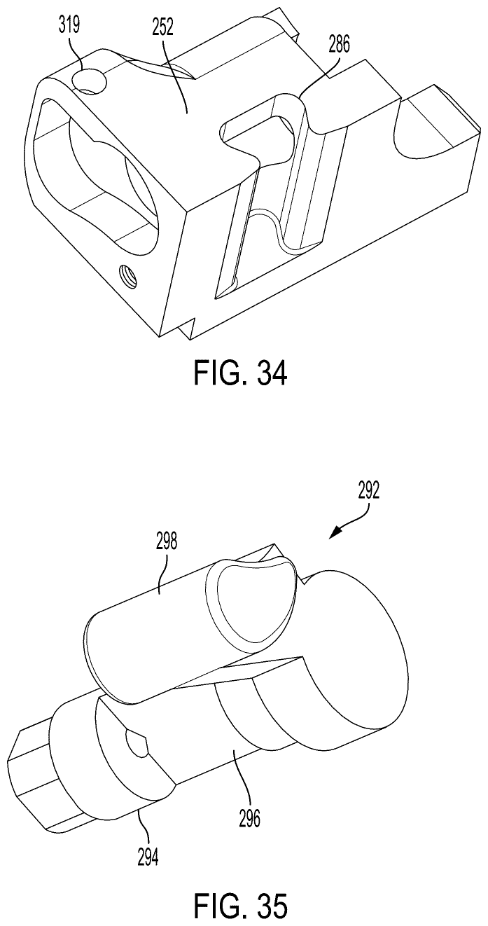

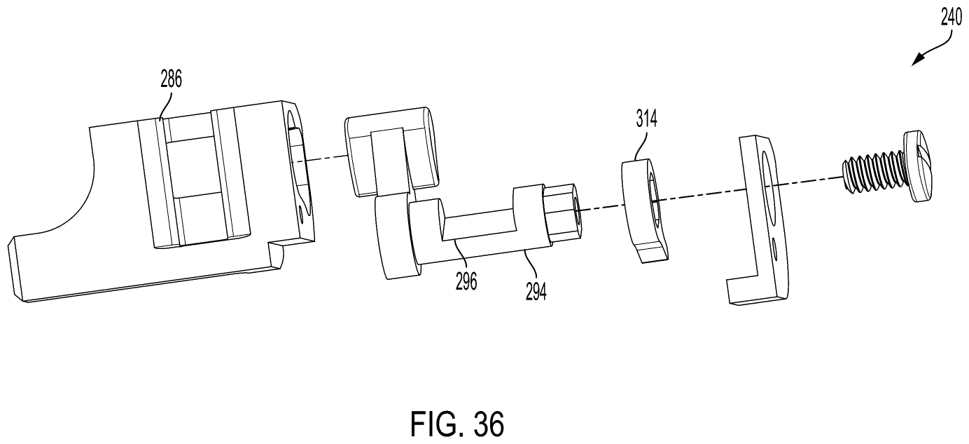

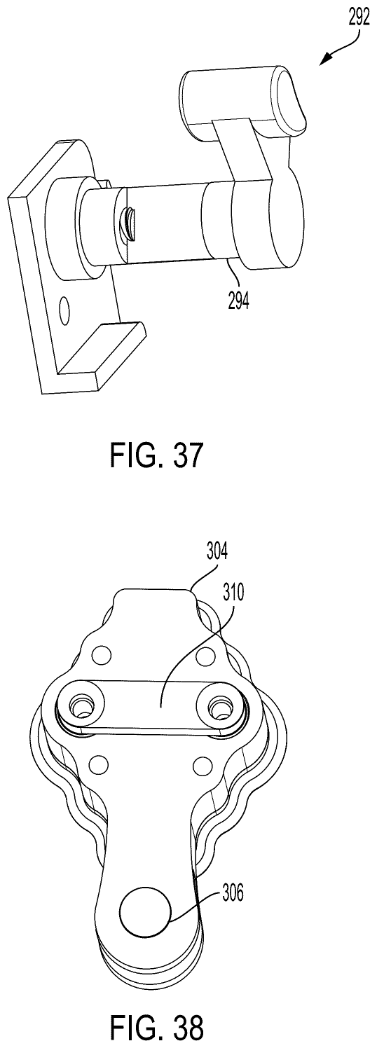

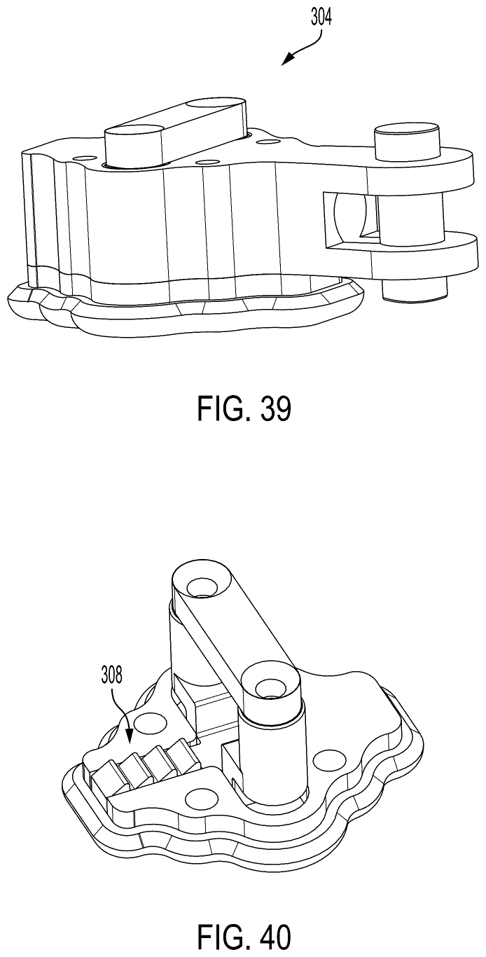

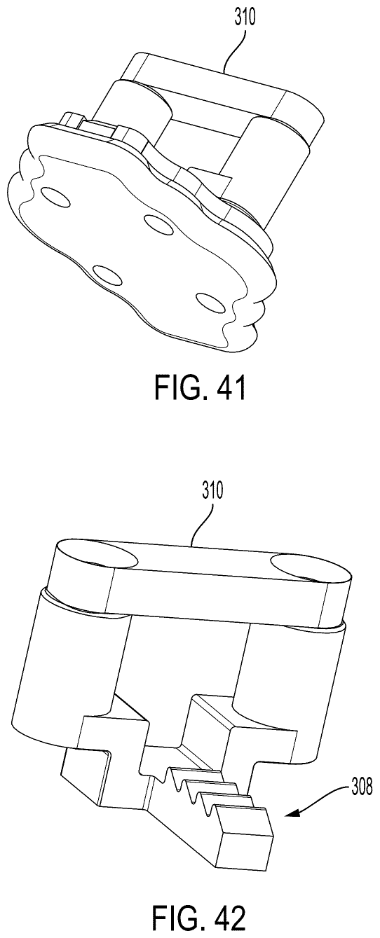

In accordance with a third exemplary embodiment, the subject disclosure provides for a surgical retractor device 210. The surgical retractor device 210 is configured as shown in FIGS. 21-43. The surgical retractor 210 includes a first retractor arm 212, a second retractor arm 214, a third retractor arm 216 and a retractor body 218.

The first retractor arm 212 is configured as best shown in FIGS. 21-24. The first retractor arm 212 is an elongated arm having a curved distal end 220, a first pivot 222 about its proximal end, and a second pivot 224 about its midportion. The first pivot 222 is positioned about its medial side or extends medially about its proximal end. The second pivot is positioned about its medial side and between the first pivot and its curved distal end. The curved distal end curves medially relative to a longitudinal axis of the first retractor arm. About a most distal end of the first retractor arm is a stub shaft 226 for engaging a first finger mount 228. The stub shaft extends distally from a substantially distally facing face of the first retractor arm.

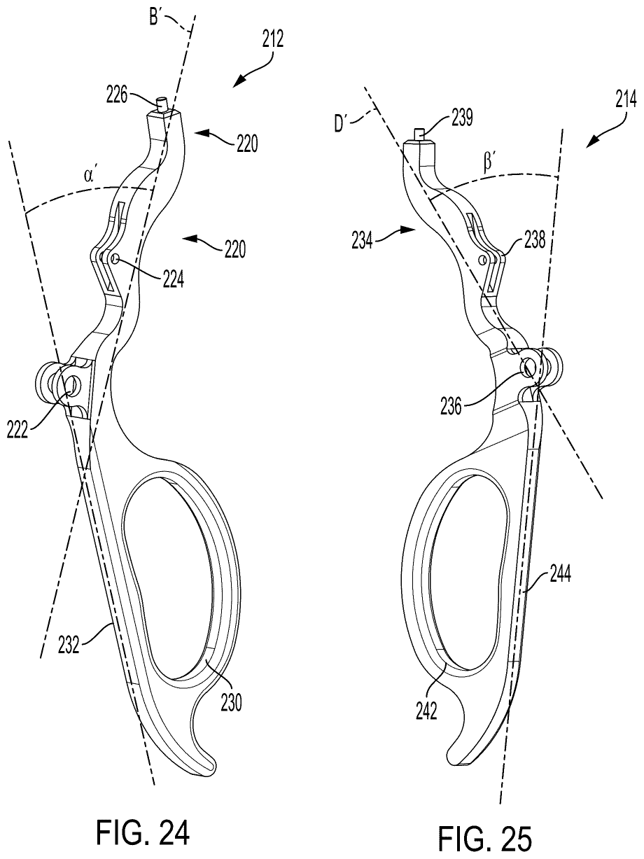

A first finger loop 230 extends from the first retractor arm. The first finger loop includes a substantially linear edge 232 with a loop of the first finger loop extending laterally from the substantially linear edge. A longitudinal axis defined by the substantially linear edge extends an angle α′ of about 15 to 45 degrees including 20, 25, 30, 30 and 40 degrees, relative to a longitudinal axis B′ of the first retractor arm (as shown in FIG. 24).

The second retractor arm 214 is configured as best shown in FIGS. 21-23, 25. The second retractor arm 214 is configured substantially the same as a mirrored configuration of the first retractor arm 212. The second retractor arm 214 includes an elongated arm having a curved distal end 234, a first pivot 236 about its proximal end, and a second pivot 238 about its midportion. The first pivot 236 is positioned about its medial side or extends medially about its proximal end. The second pivot is positioned about its medial side and between the first pivot and its curved distal end. The curved distal end 234 curves medially relative to a longitudinal axis of the second retractor arm. About a most distal end of the second retractor arm is a stub shaft 239 for engaging a second finger mount 240. The stub shaft extends distally from a substantially distally facing face of the second retractor arm.

A second finger loop 242 extends from the first retractor arm. The second finger loop includes a substantially linear edge 244 with a loop of the second finger loop extending laterally from the substantially linear edge. A longitudinal axis defined by the substantially linear edge extends an angle β′ of about 15 to 45 degrees including 20, 25, 30, 35 and 40 degrees, relative to a longitudinal axis D′ of the second retractor arm (as shown in FIG. 25).

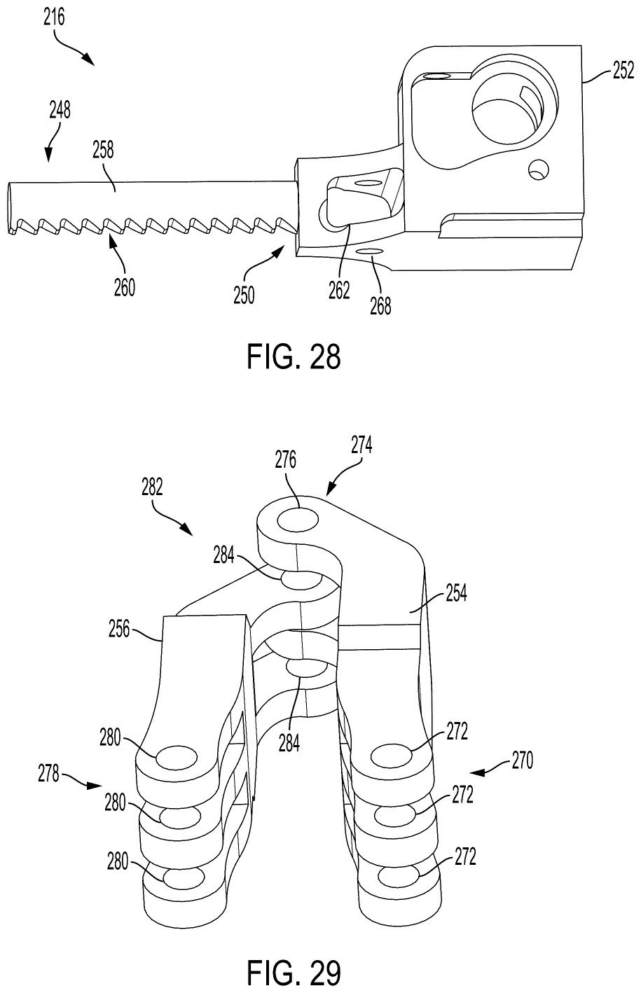

The third retractor arm 216 is configured as best shown in FIGS. 21-23 and 26-28. The third retractor arm includes an elongated body having a proximal end 248 and a distal end 250 (as shown in FIGS. 27 and 28). The third retractor arm 216 also includes a third finger mount 252, a first pivot arm 254, and a second pivot arm 256.

The proximal end of the third retractor arm 216 includes a shaft 258 having

a linear ratcheting mechanism 260 that includes ratcheting teeth.

The distal end of the third retractor arm includes the third finger mount 252 and a through cavity 262. The through cavity 262 receives, in accordance with an exemplary aspect, the first and second pivot arms 254, 256 therein. The through cavity 262 includes open lateral sides 264, 266, through hole 268 extending through the through cavity 262 transverse to the open lateral sides or alternatively transverse to a longitudinal axis of the third retractor arm.

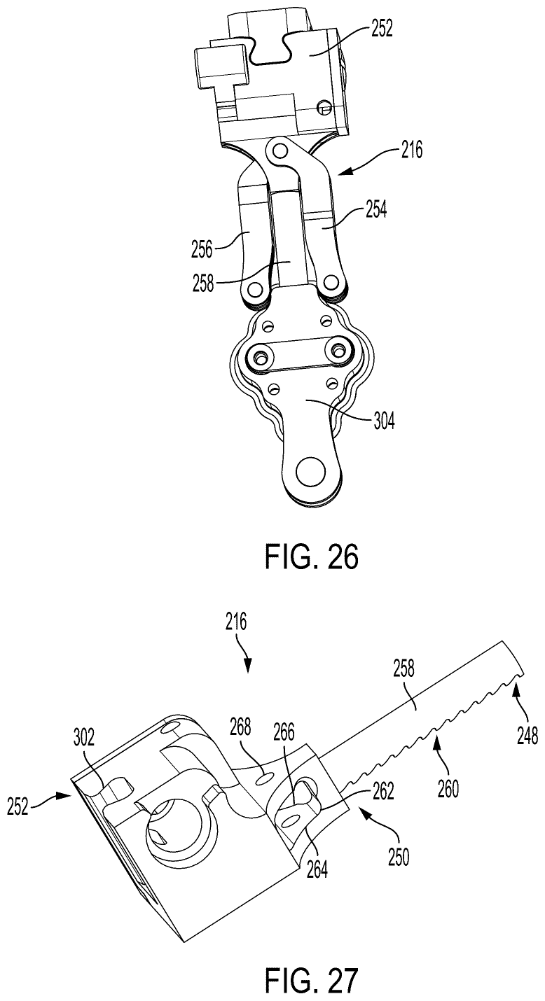

The first and second pivot arms 254, 256 are configured as best shown in FIGS. 26, 29 and 30. The first pivot arm 254 includes a proximal end 270 having a pivot connection 272 and a distal end 274 having another pivot connection 276. The pivot connections 274 and 276 can be configured as through holes for receiving respective corresponding pivots therein.

The second pivot arm 256 includes a proximal end 278 having a pivot connection 280 and a distal end 282 having another pivot connection 284. The pivot connections 280 and 284 can be configured as through holes for receiving respective corresponding pivots therein, e.g., a pivot shaft.

The first finger mount 228 is configured as best shown in FIGS. 31-37 and is attached to a distal end of the first retractor 212. The first finger mount includes a recess 286 for receiving a finger 288 therein. The recess 286 is a dovetailed recess having an open medial side or open medial face. The first finger mount also includes a lock 292 for locking the finger 288 within the dovetailed recess 286. The second finger mount 240 and the third finger mount 252 are similarly configured as the first finger mount 228.

In the present exemplary embodiment, the lock is a cam lock 292 has a cam body 294 that includes a recess 296 for receiving a tail joint 290 of a corresponding finger (as further discussed below) when assembling the finger to its respective finger mount. The cam lock 292 also includes a cam lever 298 for moving or rotating the cam lock 292 between a locked position and an unlocked position. When in the unlocked position, the tail joint 290 passes within the recess 296. To lock the finger in position, the cam lock 292 is rotated to its locked position and the cam body 294 is cammed into locking engagement with a corresponding recess 316 of the tail joint.

The cam lock also includes a spring clip 314. The spring clip 314 is a locking trigger for locking and unlocking the cam lock to the third finger mount. The spring clip 314 is attached to the non-lever end of the cam lock and secured therein via a fastener 315. The spring clip includes a locking tab 317 that engages with a recess 319 within the third finger mount for holding the cam lock in the locked position and consequently the finger within the third finger mount.

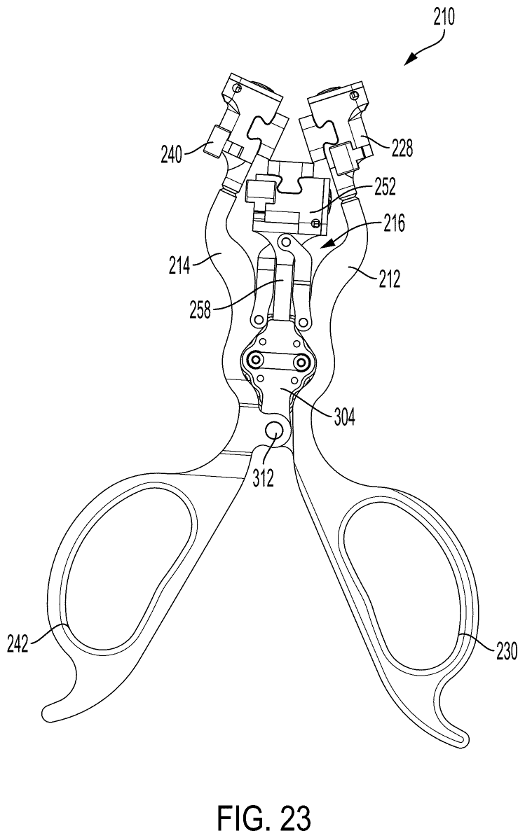

Each of the second and third finger mounts 240, 252 are similarly configured as the first finger mount 228 except for the dovetailed recess 300 of the second finger mount 240 includes an open medial side or open medial face, and the dovetailed recess 302 of the third finger mount 252 includes an open distal face or open distally facing face (as shown in FIGS. 38 and 23).

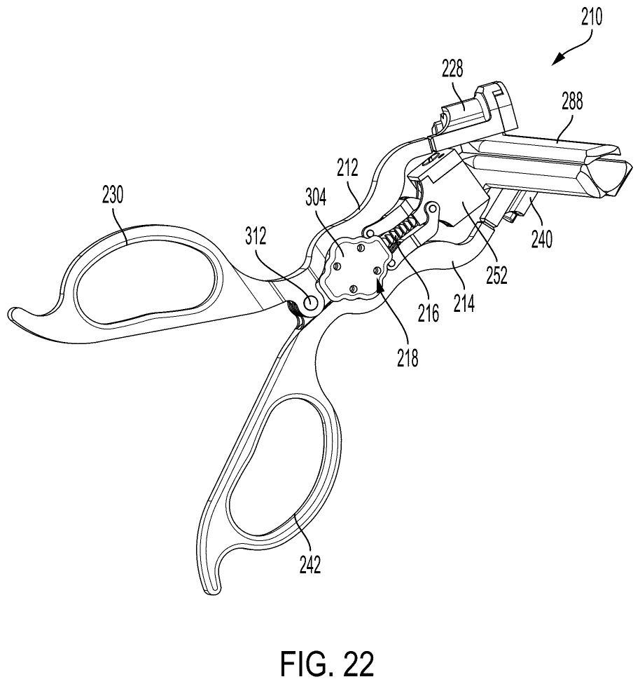

The retractor body 218 is configured as best shown in FIGS. 22-23, and 26. The retractor body includes a ratchet housing 304 and a pivot 306 about its proximal end for operatively engaging corresponding pivots 222, 236 of the first and second retractor arms 212 and 214.

Referring to FIGS. 40-42, mounted within or alternatively housed within the ratchet housing 304 is a ratcheting mechanism 308 for operatively engaging the linear ratchet mechanism 260 of the third retractor arm 216. The ratcheting mechanism 308 includes corresponding teeth for operatively engaging the linear ratchet mechanism 260. The ratcheting mechanism 308 is secured within the ratchet housing.

The ratcheting mechanism 308 also includes a button 310 extending from the ratchet housing to allow a user to lever the ratcheting mechanism 308 away from and out of engagement with the ratchet mechanism 260 so as to allow the user to move the surgical retractor 210 from the retracted position to a non-retracted position e.g., moving the ratchet mechanism relative to the ratcheting mechanism.

In some embodiments, a biasing member (e.g., a compression spring, a coil, an elastomer, and the like) may be housed within the ratchet housing to bias the ratcheting mechanism towards engagement with the linear ratchet mechanism 260. The biasing member may be positioned within corresponding cavities within the ratchet housing below the button or in a manner to bias the ratcheting mechanism towards the linear ratchet mechanism 260 e.g., upwardly.

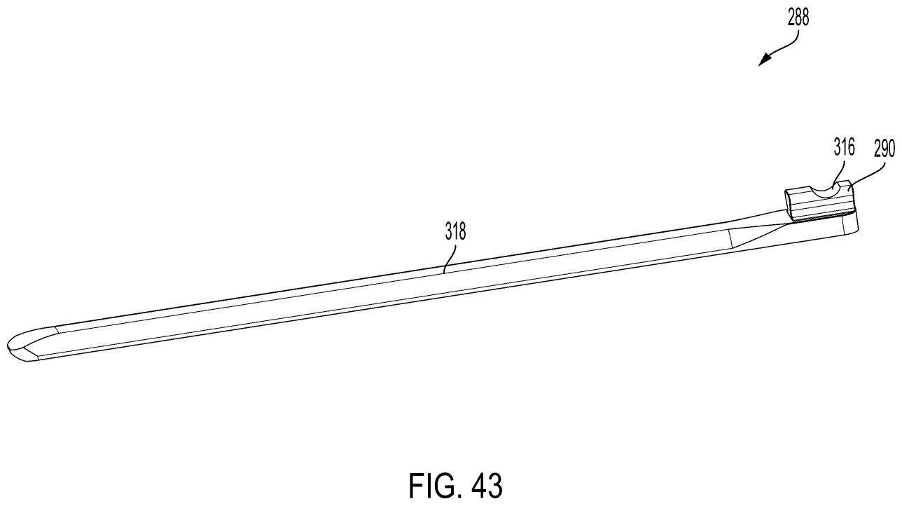

FIG. 43 illustrates an exemplary modular finger 288 applicable to the first, second, and third retractor arms. That is, finger 288 is configured to seat within the recesses of the first, second, and third retractor arms. The finger 288 is an elongated finger having a tail joint 290 correspondingly configured to seat within the dovetail recess of the first, second or third retractor arms. The tail joint includes a recess 316 sized and configured to operatively engage the cam lock 292 for securing the finger within its respective dovetail recess. The elongated finger is also configured to have a transverse cross-section that is substantially C-shaped or curved. That is, the main body 318 of the finger is curved.

Referring back to FIGS. 21-23, the first retractor arm 212 is connected to the retractor body 218 about its first pivot 222 and the second retractor arm 214 is connected to the retractor body 218 about its first pivot 236. That is the first and second retractor arms are pivotably connected to the pivot connection 312. The third retractor arm 216 is operatively connected to the retractor body 218 and more particularly operatively engages the ratchet housing 304. The first pivot arm 256 has its distal end pivotably connected to the third retractor arm 216 about its pivot connection 276 and its proximal end 270 pivotably connected to the second pivot 224 of the first retractor arm 212. The second pivot arm 256 has its distal end pivotably connected to the third retractor arm 216 about its pivot connection 284 and its proximal end 278 (via its pivot connection 280) pivotably connected to the second pivot 238 of the second retractor arm 214. Configured as such, each of the first, second and third retractor arms 212, 214 and 216 are interconnected or coupled such that movement of the first retractor arm and/or the second retractor arm drives movement of the third retractor arm. Each of the first, second and third retractor arms has a finger seated within its respective finger mounts.

In operation, owing to the configuration and structure of the first, second and third retractor arms 212, 214 and 216, the surgical retractor 210 moves between a first position or closed position and a second position or retracted position or open position. Due to the interconnected or coupled configuration of the first, second and third retractor arms, each arm moves simultaneously or in unison as a single unit. Specifically, when the user squeezes the first and second finger loops 230 and 242 together, the first and second retractor arms 212 and 214 pivot about a single axis of the pivot 312. During movement from the closed position to the open position, the first and second retractor arms rotate outwardly or away from each other, which thereby causes or drives movement of the first and second pivot arms which causes or drives movement of the third retractor arm within the retractor housing thus retracting tissue in a plurality of directions during use, e.g., in the horizontal direction (or medial lateral direction) and the vertical direction (or proximal distal direction). In the open position or retracted position, the surgical retractor is held in position by the ratcheting mechanism.

To disengage the surgical retractor 210 from the open position, e.g., to disengage the ratchet mechanism from the ratcheting mechanism, the user presses the button 310 applying a force to the ratcheting mechanism to move the ratcheting mechanism away from engagement with the ratchet mechanism. In doing so, the ratchet teeth of the respective ratcheting mechanism and linear ratchet mechanism disengage. Upon disengagement of the ratchet mechanism 260, the user can move the retractor arms (and the surgical retractor 210) back to the closed position.

The foregoing exemplary embodiments for the surgical retractor devices advantageously provides for an improved retractor. The inclusion of the third retractor arm coupled with the first and second retractor arms permits synchronized or concurrent or coupled movement, resulting in more stable, precise positioning of the device's retracting fingers with a larger field of vision. This configuration allows greater control over an incision's surgical field. By ensuring larger field of vision and consistent controllable spacing and alignment among the retractor arms, the device improves the surgeon's visibility and access. Overall, the exemplary embodiments for the surgical retractor devices enable the surgeon to achieve and maintain an improved field of vision in an easy simplified manner.

It will be appreciated by those skilled in the art that changes could be made to the exemplary embodiments described above without departing from the broad inventive concept thereof. It is to be understood, therefore, that this disclosure is not limited to the particular embodiments disclosed, but it is intended to cover modifications within the spirit and scope of the claims appended hereto.

I/we claim:

1. A surgical retractor comprising:
a first retractor arm;
a second retractor arm; and
a third retractor arm coupled with the first retractor arm and the second retractor arm to move in tandem therewith.

2. The surgical retractor of claim 1, wherein the first retractor arm and the second retractor arm pivot about a single axis.

3. The surgical retractor of claim 1, further comprising a retractor body operatively connected to the third retractor arm.

4. The surgical retractor of claim 3, wherein the retractor body includes a pivot pivotably connected to the first retractor arm and the second retractor arm.

5. The surgical retractor of claim 3, wherein the retractor body is configured to receive the third retractor arm therein.

6. The surgical retractor of claim 3, wherein the retractor body includes a ratcheting mechanism operatively engaging the third retractor arm.

7. The surgical retractor of claim 3, wherein the first, second, and third retractor arms are movable relative to the retractor body.

8. The surgical retractor of claim 1, further comprising:
a first pivot arm connected to the first retractor arm and the third retractor arm; and
a second pivot arm connected to the second retractor arm and the third retractor arm.

9. The surgical retractor of claim 1, wherein the first, second, and third retractor arms are movable between a first position and second position spaced from the first position.

10. The surgical retractor of claim 1, wherein movement of the first and second retractor arms drives movement of the third retractor arm.

11. The surgical retractor of claim 1, wherein each of the first, second, and third retractor arms comprises a modular finger extending therefrom.

12. A surgical retractor comprising:
a retractor body that includes a ratcheting mechanism;
a first retractor arm pivotably connected to the retractor body and including a first finger extending therefrom;
a second retractor arm pivotably connected to the retractor body and including a second finger extending therefrom;
a third retractor arm operatively coupled with the first and second retractor arms to move in tandem therewith and including a third finger extending therefrom, wherein the first retractor arm and the second retractor arm pivot about a single axis;
a first pivot arm connected to the first retractor arm and the third retractor arm; and
a second pivot arm connected to the second retractor arm and the third retractor arm, wherein the retractor body is configured to operatively engage the third retractor arm.
